(19) United States
(12) Patent Application Publication
(10) Pub. No.: US 2003/0027803 A1
(43) Pub. Date: Feb. 6, 2003

(54) Method and composition for inhibiting the incidence and proliferation of nervous system and brain cancer cells

(75) Inventors: Thomas J. Slaga, Golden, CO (US); Addanki P. Kumar, Lakewood, CO (US); William Alworth, New Orleans, LA (US)

Correspondence Address: DAVID G. HENRY, 900 Washington Avenue, P.O. Box 1470, Waco, TX, 76701, US

(73) Assignee: Oncology Sciences Corporation, Austin, TX (US)

(21) Appl. No.: 09/777,559

(22) Filed: Feb. 6, 2001

Related U.S. Application Data
(63) Continuation-in-part of application No. 09/527,283, filed on Mar. 17, 2000, now abandoned.
Application No. 09/527,283 is a continuation-in-part of application No. 09/777,151, filed on Feb. 5, 2001.

(51) Int. Cl. A61K 31/58; A61K 31/56

(52) U.S. Cl. 514/174; 514/182

(57) ABSTRACT

The invention is of both a composition and method for inhibiting the proliferation of cancerous cells, and brain and nervous system cells in particular. The composition is, and the method is based on the use of a composition consisting (among active ingredients) substantially of 2-methoxyestradiol and/or one of a number of analogues thereof. The present inventors have demonstrated beyond serious doubt that these compounds have a pronounced effect in inhibiting the proliferation of cancerous brain and nervous system cells and, therefore, provide a desperately needed stepping stone for advancing toward meaningful treatment of brain and nervous system cancer.

### CITATION TO PRIOR APPLICATION

This is a continuation-in-part with respect to U.S. application, Ser. No. 09/527283, filed Mar. 17, 2000 from which priority is claimed under 35 U.S.C. §120 and under provisions of the Patent Cooperation Treaty. This is also a continuation-in-part with respect to that prior application (filed Feb. 5, 2001—no serial number yet available) which also claims priority of Ser. No. 09/527283.

### BACKGROUND OF THE INVENTION

A. Field of the Invention

The present invention relates to non-surgical intervention of brain and nervous system cancer, as well as prophylactic use of herein identified compounds in the prevention of brain and nervous system cancer.

B. Background of the Invention

1. The Problem: Brain Cancer

Metastatic and primary brain and nervous system (hereinafter “NS”) cancer affects the lives of approximately 185,239 people in the United States a year. This year approximately 13,000 people in the United States alone will die of malignant brain tumors-that is more than 35 people a day. Unfortunately, a group particularly victimized by this disease are children. Brain tumors are the second leading cause of cancer-related deaths in children, accounting for approximately 25% of all of such deaths. Alarmingly, brain and NS cancer has continued to increase in both incidence and mortality in the United States. Presently, for example, 80% of patients with medulloblastoma, which represents about 25% of childhood brain tumors, currently die within the first year of diagnosis.

Brain and NS cancer can arise in various forms in the diverse structures of the brain and nervous system. The most prevalent forms of the disease are: primitive neuroectodermal tumors/medulloblastoma (ages 0-9), astrocytoma (ages 10-14), pilocytic astrocytoma (ages 15-19), pituitary tumors (ages 20-34), meningiomas (ages 35-44), glioblastoma (ages 45-74), and meningioma (ages 75 and older). Whether the end result of the disease is death or not, the disease can have profound and catastrophic effects on individuals and their ability to perceive and function in the world. The disease often robs people of their intelligence, memory, consciousness, emotions, and/or basic motor functions. The disease leaves people limited or unable to perceive the world around them, unable to perform or remember simple tasks, otherwise grossly impaired, and/or dead.

Currently, the standard treatment for such tumors comprises radiation treatment, chemotherapy, surgery, or a combination thereof. There are serious limitations and dangers associated with all of the current methods of treatment. Radiotherapy, which often attempts to deliver highly destructive doses of ionizing radiation through the normal tissues of the body in an attempt to preferentially kill highly specific and often imperfectly defined areas of cancerous tissue, can have serious and significant side effects due to the destruction of normal nervous system or other tissues of the body. Chemotherapy, which attempts to preferentially kill cancerous cells instead of normal cells through the diverse administration of chemical agents or drugs to the tissues of the body, is limited in efficacy by the chemical agents currently available and can lead to toxic and unintended side effects on normal tissue. Surgery, which attempts to mechanically destroy or intervene in the progression of cancer, can also lead to serious side effects or consequences as a result of mechanical trauma or destruction of normal tissue. Some of the problems associated with the above approaches are (i) adverse side effects including alterations of intelligence, learning ability, memory, motor function, consciousness, and emotion; (ii) re-emergence of the tumor within three to five years of treatment due to development of resistance to these therapies; (iii) death due to the ineffectiveness of such treatment.

One of the problems that characterize a number of solid brain and nervous system cancers is the unregulated growth or unchecked life span of aberrant cells in the tissues of the brain and nervous system. Normal cells grow, divide, and die on a regular basis. The process by which cells normally die is called apoptosis. However, when normal cell growth and death become unchecked in the body, by any number of processes, such unchecked growth and/or immortality leads to the formation of cancerous tumors or cell populations that can interfere and ultimately destroy the regular functioning of the tissues of the body. Such growth or immortality can ultimately lead to the occurrence of a host of solid tumors or the metastasis of cancer cells throughout the body. Unchecked cell growth and/or immortality are problematic biological mechanisms common to almost all types of brain and nervous system cancer.

Another biological mechanism that is common to, and problematic in the treatment of, all solid brain and nervous system cancer tumors is angiogenesis. Angiogenesis refers to the process by which new blood vessels are formed in the body. Without a dedicated blood supply, solid tumors have only limited growth potential—perhaps 2 mm in diameter maximum. However, angiogenesis often occurs in cancerous tissues and tumors, thus enabling solid tumors to sequester greater amounts of nutrients from the body and allowing them to proliferate rapidly, even spreading to other parts of the body. Angiogenesis is a critical means by which solid tumors grow rapidly and metastasize, hastening the process of death or disfigurement.

These two independent biological mechanisms are the common, primary modalities by which brain and nervous system cancer cells proliferate and grow. Hence, a novel approach for the treatment of brain and nervous system cancer would be the development of pharmacological agents that have dual roles as anti-angiogenic as well as pro-apoptotic agents. Such an agent will have the ability to target both components of a cancer: kill the tumor cell by induction of apoptosis and cut off the blood supply to the tumor cell so that it will not grow.

Therefore, there exists an urgently compelling, yet unsatisfied need to develop strategies for the development of a class of compounds that have both anti-angiogenic as well as pro-apoptotic properties. Additionaly, there exists an urgently compelling, yet unsatisfied need to develop strategies for the treatment, management, and ablation of brain and nervous system cancer.

2. One Solution: 2-methoxyestradiol (2-ME) and its Derivatives

A recent breakthrough in the treatment of cancer is the use of 2-methoxyoestradiol (hereinafter “2-ME”). 2-ME is an endogenous non-toxic metabolic byproduct of estrogens that is present in human urine and blood. (1) A potential role for 2-ME as a chemopreventive agent has been reported in the mammary and pancreatic models. (2) 2-ME has also been shown to inhibit endothelial cell proliferation implicating its potential role in angiogenesis. (3) In addition, apoptosis has been implicated as a mechanism for 2-ME's cytostatic and anti-angiogenic effect. The present inventors work, filed with the original patent application, shows that 2-ME is of great significance in the treatment of prostate cancer through the induction of apoptosis. This body of work indicates that 2-ME is an anti-tumorigenic agent with a significant therapeutic advantage since it can preferentially inhibit actively proliferating cells (characteristic of tumor cells) without affecting the growth of normal cycling cells. Additionally, 2-ME appears to also inhibit the formation of new blood vessels. To the best of our knowledge, this is the first compound that targets two components of cancer: the tumor cells and their blood supply. From these facts, the present inventors postulated that 2-ME may aid people with brain and/or nervous system tumors, either by shrinking the tumors or by slowing their spread (metastasis) or by cutting the blood flow to the tumor (angiogenesis). The work herein demonstrates that 2-ME is a chemical compound with a significant role as an antitumorogenic agent with broad efficacy in brain and nervous system cancer.

Building on these findings, further experiments have helped to elucidate the structural bases for 2-ME's molecular efficacy. A number of experiments have been conducted using 2-ME and 16-epiestriol (hereinafter “16-ES”), an analogue of 2-ME that lacks the methoxy group at the second position. In a multitude of experiments, using prostate cancer cell lines (both androgen-dependent (LNCaP), and androgen-independent (DU145) cells), and a brain and/or nervous system cancer cell line (DAOY), the present inventors have studied the effects of 2-ME and 16-ES on cell proliferation and the induction of apoptosis, in a number of ways. The sum of all the data clearly indicates that 2-ME is a compound that significantly inhibits cancerous cell growth and has proapoptotic effects, while 16-ES does not. In total, these data suggests that the efficacy of 2-ME may be associated with the methoxy moiety at the second position of 17β-estradiol (E₂). Further, it also suggests the possible efficacy of a series of compounds with various moieties at the second position in the treatment of cancer. Additionally, the specific anti-proliferative, pro-apoptotic, anti-angiogenesis, and other efficacy of 2-ME against cancer cells suggests that other structural modifications of the molecule should be explored in attempts to increase the efficacy of the agent. From these facts, the present inventors postulated that analogues of 2-ME may aid people with brain and/or nervous system tumors, either by shrinking the tumors or by slowing their spread (metastasis) or by cutting the blood flow to the tumor (angiogenesis). The work herein demonstrates that analogues of 2-ME are chemical compound with a significant role as an antitumorogenic agent with broad efficacy in brain and nervous system cancer.

### SUMMARY OF THE INVENTION

It is an object of the present invention to provide a composition which is efficacious in inhibiting the proliferation of brain and nervous system cancer cells.

It is another object of the present invention to provide a composition the primary active ingredient of which are an analogue or analogues of 2-methoxyestradiol which are efficacious in inhibiting the proliferation and/or angiogenesis of brain and nervous system cancer cells.

It is another object of the present invention to provide a method for inhibiting the proliferation and/or angiogenesis of brain and nervous system cancer cells through use of a composition the primary active ingredient of which is 2-methoxyestradiol or an analogue thereof, as described herein.

In satisfaction of these and related objectives, the present invention provides both a composition and method for inhibiting the proliferation of brain and nervous system cancer cells. The composition is, and the method is based on the use of a composition consisting (among active ingredients) substantially of 2-methoxyestradiol and/or a number of analogues thereof. The present inventors have demonstrated beyond serious doubt that these compounds have a pronounced effect in inhibiting the proliferation of cancerous brain and nervous system cells and, therefore, provide a desperately needed stepping stone for advancing toward meaningful treatment of brain and nervous system cancer.

### DETAILED DESCRIPTION OF THE PREFERRED EMBODIMENT

I. Efficacy of 2-ME In Brain and Nervous System Cancer

The present inventors have tested the use of 2-methoxyestradiol (Hereinafter “2-ME”) in brain and nervous system cancer prevention in an in vitro system, and have determined that there exists proven efficacy in this regard. This testing has involved the use of cell lines from two of the most common brain and nervous system cancers, medulloblastoma (DAOY) and astrocytoma (T98-G and U97-MG), in order to investigate the effect of 2-ME. Also utilized was the non-tumorigenic cell line U87 for the same purposes. The cell lines DAOY, U87, and T98-G were obtained from Dr. Victor Levin at The University of Texas M.D. Anderson Cancer Center in Houston, Tex.

### Cell Culture

The cells were treated with different concentrations of 2-ME (0 to 10 μM) and cell growth, cell morphology, cell cycle progression, DNA laddering, caspase-3 activity, and p53 activity were monitored in order to evaluate the efficacy and function of 2-ME in brain cancer cells.

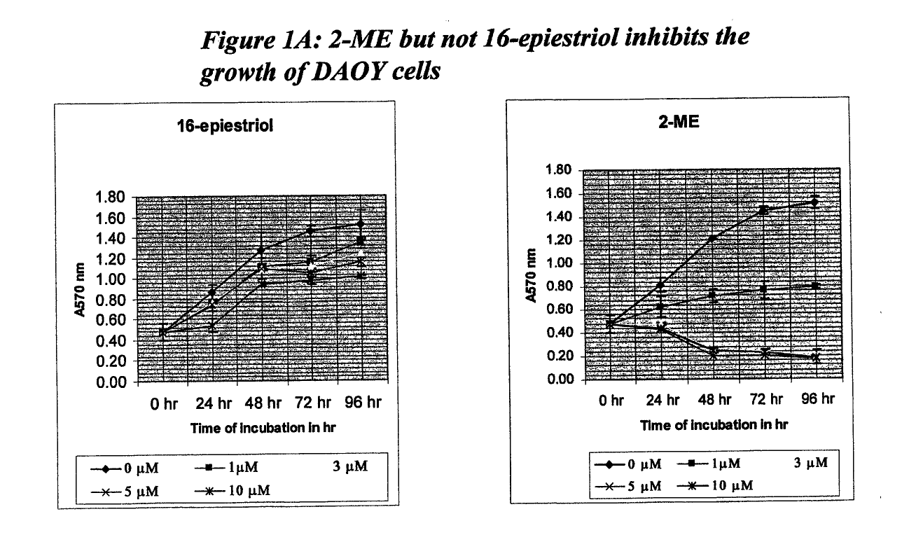

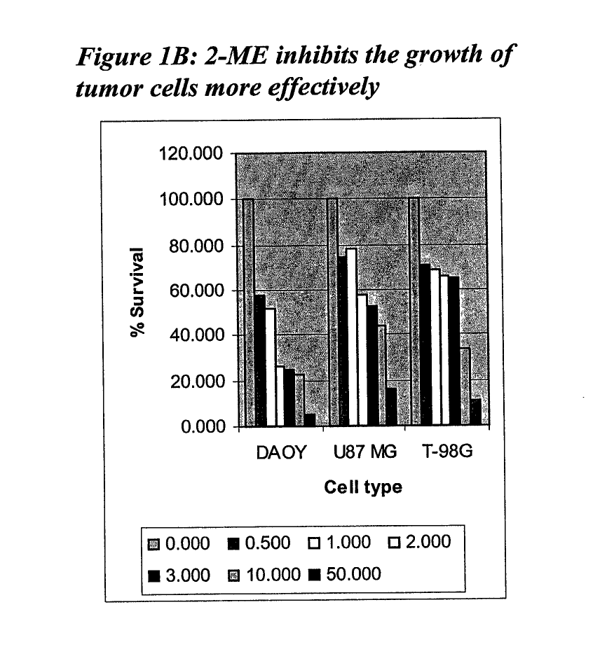

FIG. 1 (A & B): Cell Growth

Referring to FIG. 1, actively growing DAOY cells were plated in 96-well plates at a density of 4×10³ cells per well in five replicates. After 24 hours in a 37 degree Celsius incubator with 5% CO₂, the cells were treated with the indicated concentration of 2-ME. Control cells received only the vehicle (DMSO). Cell viability was determined by the tryphan blue exclusion assay. Cell growth was monitored every 24 hours using the CellTiter96 Aqueous One solution assay containing a tetrazolium compound (Promega Corporation, Madison, Wis.).

The assay is based on the principle that actively growing cells generate reducing equivalents such as NADH that is necessary for the cells to reduce the tetrazolium compound to a formazan product. This was detected by measuring the absorbance at 570 nm using Molecular Devices SpectraMaxPlus plate reader. An increase in the conversion of the MTS tetrazolium compound to the colored formazan product indicated by an increased absorbance provides a relative measure of viable cell number, while a decrease provides a relative measure of cell death.

The present inventors examined the effect of 2-ME or 16-episteriol on cell proliferation of DAOY cells using the CellTiter96 Aqueous One solution assay as described earlier. (4) Exponentially growing DAOY cells were treated with 0, 1, 3, 5, and 10 μM of 2-ME or 16-episteriol (Hereinafter “16-ES”) and cell growth was measured every 24 hours as previously described. 16-ES is an analog of 2-ME that lacks the methoxy group at the second position. As shown in FIG. 1A, 2-ME at 3 μM exhibited 50% inhibition of growth in 24 hours. In contrast, the control cells or the cells treated with 16-ES continued to proliferate during the course of the experiment. This data indicates that 2-ME is a potent inhibitor of proliferation of DAOY cells and the observed inhibitory effect is specific to 2-ME. Since 16-epiestriol is an inactive analog of 2-ME that lacks the methoxy group at the second position, these results also indicate the importance of the methoxy moiety for the observed growth inhibitory potential of 2-ME. These results were also confirmed by measuring cell viability using the trypan blue exclusion method (data not shown) in our lab.

In order to determine the specificity of 2-ME towards tumor cells, the brain and NS cancer cell line DAOY and the non-tumorigenic cell line T98-G were treated with 2-ME. Although the growth was reduced in both cell lines in response to 2-ME treatment, it was more effective in DAOY cells. For example, 2 μM of 2-ME decreased the growth of DAOY cells by about 80% after 72 hours of incubation (FIG. 1B). In contrast, under similar experimental conditions, the growth of T-98G cells was decreased by only about 38%. In order to produce comparable effects, a concentration of more than 10 μM was necessary. These results indicate the specificity of 2-ME towards tumor cells.

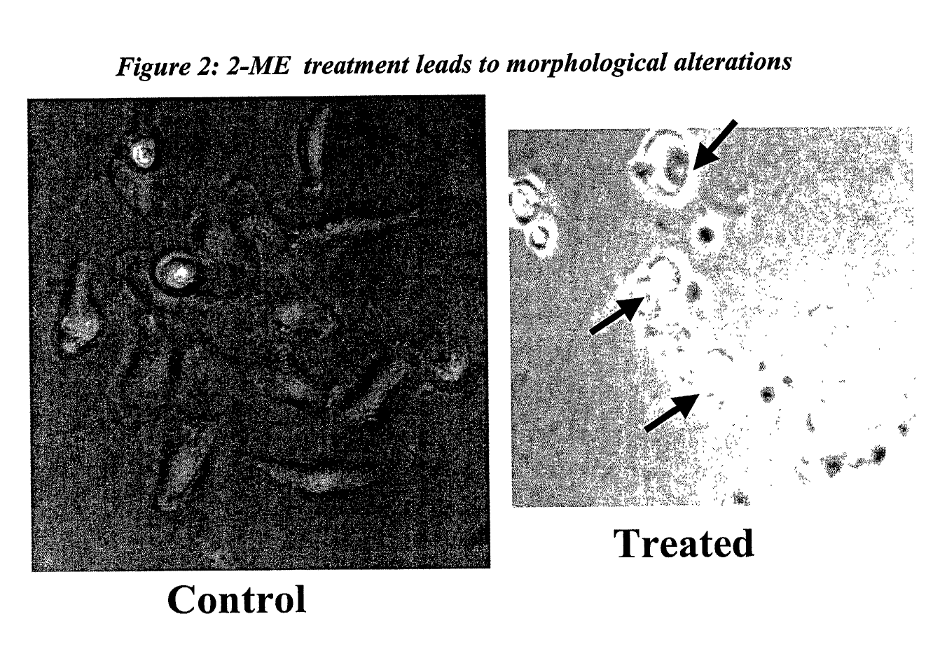

### FIG. 2: Morphological Response

Exponentially growing DAOY cells at 70-80% confluence were treated with 2-ME (3 μM). The cells were observed every 24 hours for any morphological changes associated with 2-ME treatment. As shown in FIG. 2, following treatment, cells showed significant morphological alterations: cells became rounded, shrank, retracted from their neighboring cells, the cytoplasm became condensed and a significant proportion of the cells started to float by 24 hour and finally detached from the dishes. These observations indicate that a significant proportion of cells exhibited apoptotic features following 2-ME treatment. Similarly, under identical conditions, DAOY cells were exposed to the vehicle (DMSO) or 16-ES. Unlike the 2-ME treated DAOY cells, the DMSO and 16-ES treated cells did not exhibit any of the morphological changes associated with apoptosis. To determine the mechanism of growth inhibition by 2-ME, alterations in cell cycle distribution following 2-ME treatment was examined.

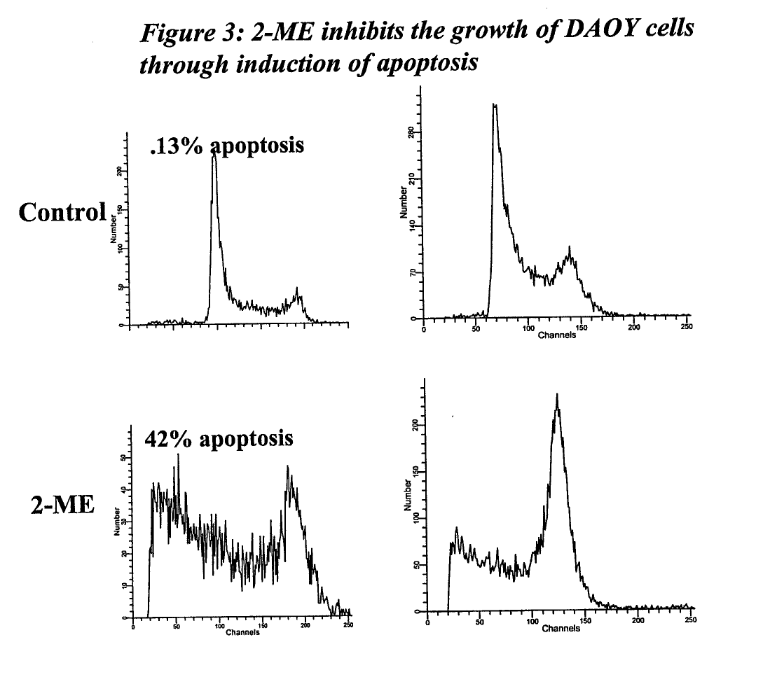

### FIG. 3: Flow Cytometry

Logarithmically growing cells were plated at a density of 10⁵ cells in 60 mm dishes as described above. Exponentially growing cells, at 70-80% confluence, were treated at time 0 with 3 μM 2-ME or 16-ES for 24 hours. Cells were harvested by trypsinization and the cell pellet was re-suspended in 1 ml of Krishan stain containing 1.1 mg/ml of sodium citrate; 46 μg/ml of propidium iodide; .01% of NP40 and 10 μg/ml of RNAse (5) and subjected to flow cytometric analysis at the Flow Cytometry core facility of the University of Colorado Comprehensive Cancer Center, Denver, using a Coulter XL flow cytometer (Beckman-Coulter, Hialeah, Fla.). Data was analyzed using Modfit LT from Verity Software House (Topsham, Me.).

In order to determine the mechanism of growth inhibition by 2-ME, the effect of 2-ME on cell cycle distribution by FACS analysis was studied. As shown in FIG. 3, flow cytometric analysis of DAOY cells treated with 3 μM of 2-ME for 24 hours showed approximately a 2-4 fold increase in the G2/M population with a concomitant decrease in population of cells in G1 and S phase. In addition, a sub-G1 peak was observed, suggesting the presence of apoptotic cells with fragmented DNA (FIG. 3A). (6) Staining of cells with Saponin showed about 22-42% of the cells were undergoing apoptosis (FIG. 3B). However, treatment of cells with 16-ES had no significant effect on the distribution of cells. This data suggests that 2-ME inhibits the growth of DAOY cells (i) by arresting the cells predominantly in the G2/M phase; (ii) possibly through induction of apoptosis.

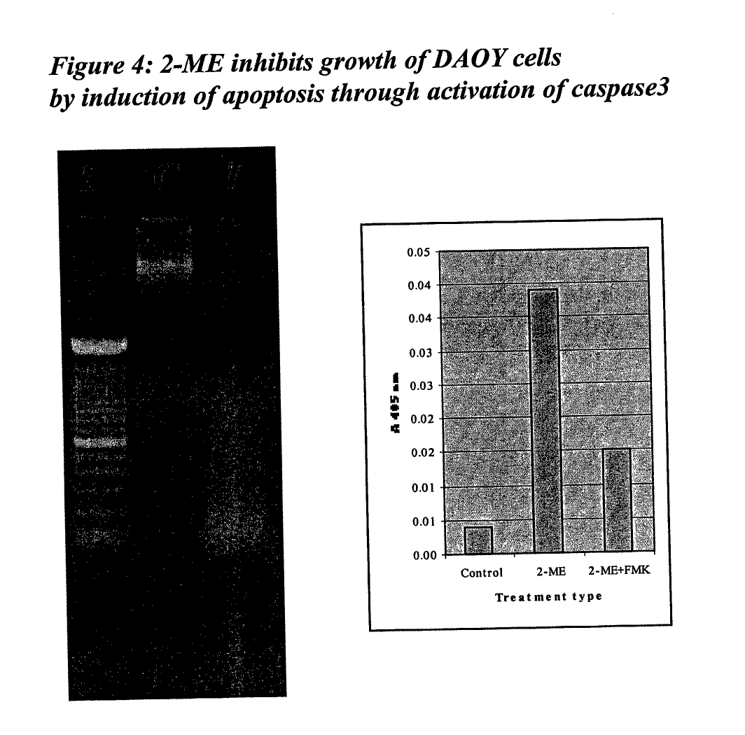

### FIG. 4: Apoptosis/Activation of Caspase-3

FIG. 4A: Induction of apoptosis by 2-ME was detected using the ApoTarget Quick Apoptotic DNA ladder detection kit from BioSource International, Inc. (Camarillo, Calif.). Genomic DNA was isolated from the cells treated with 3 μM 2-ME for 24 hours, following the manufacturer's directions. DNA concentration was estimated by measuring the absorbance at 260 nm and equal amounts of DNA were loaded onto a 1% agarose gel.

Since the above cell cycle data indicated induction of apoptosis as a potential mechanism for 2-ME's antiproliferative activity, the issue of whether 2-ME treatment could induce apoptosis in brain and nervous system cancer cells was explored. Apoptosis is an active, genetically controlled process by which cells self-destruct and is accompanied by characteristic morphological and biochemical changes. (7) One of the earliest events during this process is the activation of a calcium-dependent endonuclease associated with nuclear DNA fragmentation. (8) During this process chromosomal DNA is degraded primarily into large DNA fragments followed by subsequent formation of smaller oligonucleosomal fragments resulting in the appearance of a defined DNA ladder when analyzed by agarose gel electrophoresis. Genomic DNA was prepared from DAOY cells following treatment with 2-ME using the ApoTarget DNA ladder detection kit according to the manufacturer's protocol. (BioSource International, Inc., Camarillo, Calif.) As shown in FIG. 4A, multiple DNA fragments, or laddering, resulting from internucleosomal cleavage, were clearly visible in DAOY cells treated with 2-ME for 24 hours. In contrast, no evidence of DNA laddering was detected in the control cells. Thus, the ability of 2-ME to inhibit the growth of these cells is due in part to 2-ME's induction of apoptosis.

FIG. 4B: To corroborate the findings with regards to apoptosis, the present inventors also utilized the CaspACE Assay System (Promega Corporation, Madison, Wis.) to measure the DEVDase activity. Increase in the activity of DEVDase (caspase 3) is an early regulatory event in the induction of apoptosis. The caspase activity system measures the release of p-nitroaniline (hereinafter “p-NA”), a yellow colored product from the substrate Ac-DEVD-pNA colorimetrically. Exponentially growing cells at 70-80% confluence were treated at time 0 with 3 μM of the following for 24 hours: (i) 2-ME-, (ii) 16-ES, or (iii) 2-ME and, an inhibitor of caspase 3, Z-VAD-FMK. Following this incubation, cell extracts were prepared according to the manufacturer's protocol and protein content of the extracts was determined by the method of Bradford. Equal amounts of protein were used in determining caspase activity. (9)

Activation of a series of cytosolic proteases called caspases has been shown to be involved in the induction of apoptosis by a wide variety of agents and extracellular signals resulting in the cleavage of various cellular protein substrates leading to impairment of tissue homeostasis and ultimate destruction of the cell. (10) Among these proteases, caspase-3 has been implicated as a key protease that is activated during the early stages of apoptosis and is detected only in cells undergoing apoptosis. As shown in FIG. 4B, no caspase activity was detected in the extracts prepared from the untreated or 16-ES treated cells. In contrast, caspase-3 activity was detected in extracts prepared from cells treated with 3 μM 2-ME. This 2-ME induced caspase activity was reduced by approximately 64% when extracts were prepared from cells treated with both 2-ME and Z-VAD-FMK, a caspase inhibitor, indicating that 2-ME's activity is mediated through caspase-3. This data suggests that 2-ME probably inhibits the growth of DAOY cells by inducing apoptosis that is mediated by activation of caspase-3. Taken together, these results demonstrate that 2-ME inhibits the growth of DAOY cells through the induction of apoptosis.

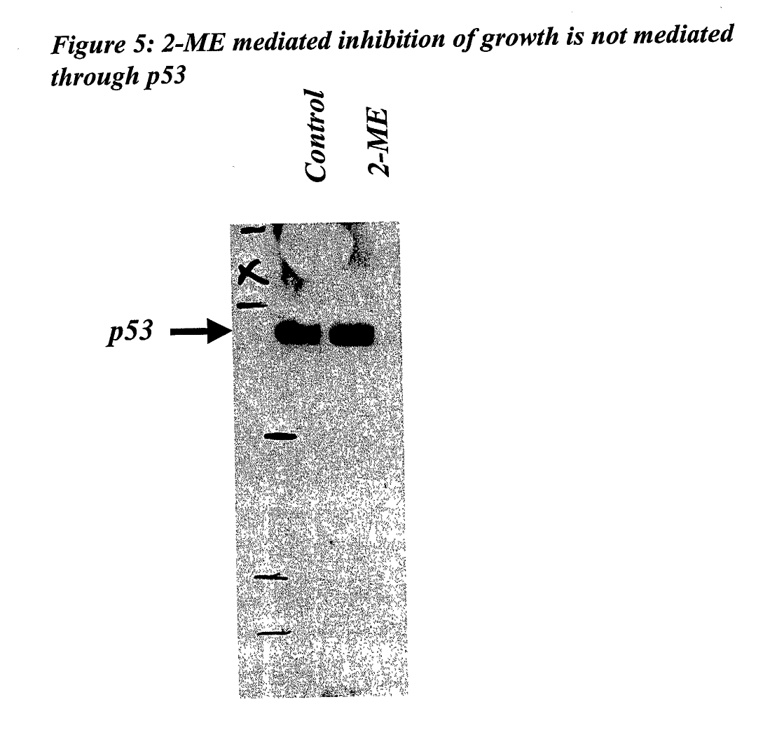

### FIG. 5: p53's Involvement

Cells were grown as described above and treated with 3 μM 2-Me for 24 hours. Following treatment, cell extracts were prepared by lysing the cells in a buffer containing: 50 mM Tris-HCl (pH 7.4), 150 mM NaCl, 0.5% NP40, 50 mM NaF, 1 mM NaVO₄, 1 mM phenylmethylsuflonyl fluoride, 25 μg/ml of leupeptin, 25 μg/ml aprotinin, 25 μg/ml pepstatin, and 1 mM DTT. After passing the lysate through a 25 gauge needle, cell debris was removed by centrifuging the lysate at 12000 rpm for 30 minutes. Protein content of the extracts was determined by the method of Bradford. (9)

Equal amounts of extracts were fractioned on a 10% SDS-polyacrylamide gel. (11) Following electrophoresis, proteins were electrophorectically transferred to nitrocellulose membrane. The blotted membrane was blocked with 5% non-fat dried milk in Tris-buffered saline containing 0.1% Tween 20 (blocking solution) and incubated with polyclonal p53 antibody (Santa Cruz Biotechnology, Calif.) followed by incubation with horse radish peroxidase-conjugated anti-rabbit IgG antibody (Sigma, ??) in blocking solution. [Clarify—please be more specific] Bound antibody was detected by enhanced chemiluminescence using Supersignal West Pico Chemiluminescent Substrate, following the manufacturer's protocol. (Pierce, Rockford, Ill.)

It is believed that the initiation of apoptosis can take place at any phase in the cell cycle. The flow cytometric data demonstrated that 2-ME's effect was to arrest the brain and nervous system cancer cells at the G2/M phase, which was then followed by cell death. The tumor suppressor protein p53 is a key regulator of cell cycle check point and can induce apoptosis in a wide variety of experimental systems through distinct pathways. (12-15) It is also well established that the tumor suppressor function of p53 is mediated by accumulation of wild-type p53 in response to extracellular signals, with the sequential induction of either cell cycle arrest or apoptosis. (12) Mutation of p53, which occurs in approximately 50% of human cancers, is the result of the disruption of these signaling pathways. It is commonly thought that the deregulation of such signaling pathways is what ultimately provides tumor cells with a selective growth advantage, compared to normal tissues. In order to test the hypothesis that the observed 2-ME induced growth inhibition was mediated by alterations in the levels of p53, control and 2-ME treated cells were prepared as above and were examined for p53 expression using western blot analysis.

As shown in FIG. 5, significant changes in the levels of p53 expression following treatment were not observed. This data indicates that the observed apoptosis following 2-ME treatment may not be mediated through p53. However, one cannot rule out the participation of other members of the p53 family, including p73 and p51, in mediating 2-ME's growth inhibitory effect. (12) Interestingly, all these members can bind to the p53 consensus DNA binding sequence and activate transcription and also induce apoptosis, irrespective of p53 status. (13-15) Alternatively, a p53-independent pathway may be involved.

Overall, these results suggest that 2-ME can be used as a potential therapeutic agent for the treatment of brain and nervous system cancer with numerous advantages: (i) it is a natural endogenous estrogen metabolite that reaches micromolar levels during pregnancy, indicating it's non-toxic nature; (ii) it has a dual advantage of being able to inhibit cell proliferation and formation of new blood vessels necessary for tumor cell proliferation and metabolism. The latter property is especially beneficial in the treatment of brain tumors, as one of the limitations of treating brain tumors is the blood brain barrier. Although the sensitivity of primary or normal cells to 2-ME is not know, the differential sensitivity of 2-ME towards the different cell lines used in this study suggests that it may have no effect on normal cells. Unraveling the mechanisms of 2-ME induced apoptosis may lead to more effective medical therapy for brain tumors, reduction in mortality and morbidity following conventional therapy.

II. 2-ME Analogues

Data from the present inventors laboratory shows that 2-ME inhibits the growth of brain and nervous system cancer cells but that 16-epiestriol does not. This indicates that substituting the second position of 17b-estradiol (E₂) with a methoxy group generates a molecular structure that shows significant and selective growth inhibitory activity toward brain and nervous system cancer cells while simultaneously eliminating the potentially detrimental growth stimulating activity of E₂ itself. The analogues of 2-ME to be prepared as described below are designed (1) to determine which components of the 2-ME molecule in addition to the 2-methoxy group are required for the observed chemopreventive effects and (2) to determine if growth-inhibitory 2-ME analogues can be created that are effective in the treatment of brain and nervous system cancer.

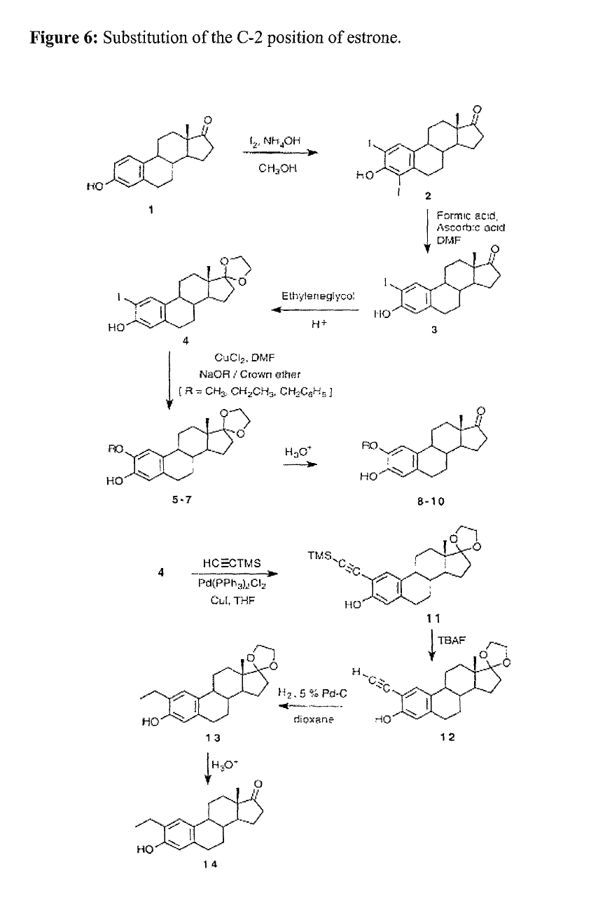

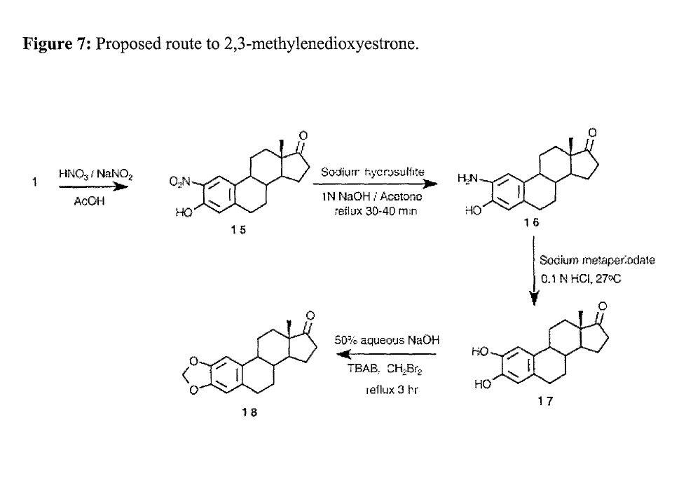

The initial compounds to be synthesized will be 2 alkoxy substituted analogues of estrone shown in FIG. 6. These compounds will then be converted into the 2-ME analogues as shown in FIG. 3 (analogues 19-21, 23-25, and 27-29).

FIG. 6 illustrates how the A ring of the E₂ steroidal nucleus will be modified to generate 2-alkoxy substituted analogues of estrone (analogues 8-10) and a 2-ethyl substituted estrone analogue (analogue 14). The key reactions in this figure are the synthesis of compound 2, 2,4-diiodoestrone, and its conversion to compound 3, the 2-iodoestrone derivative. The iodination and diodination of the estrone starting material (analogue 1) will be carried out as described by Ikegawa et al in their synthesis of catecholic equilin and equilin derivatives. (16) The proposed conversion of the ethylenedioxy protected 2-iodoestrone derivative 4 to the protected 2-methoxy, 2-ethoxy, and 2 benzyloxy derivatives 5-7 by Cu (I) catalyzed reactions of the alkoxides in dimethylformamide in the presence of a crown ether is based upon the comparable reaction of a protected 2-iodoequilin also described by Ikegawa et. al in the synthesis of catechol equilins. (16) It should be noted that if it proves necessary the estrone starting material used in FIG. 6 could be protected as the ethylenedioxy derivative by treatment with ethylene glycol prior to the iodination reaction. The Pd(Ph₃)Cl₂/CuI catalyzed coupling of the aryl iodide (analogue 4) with trimethylsilyl substituted acetylene to yield the 2-alkynyl substituted estrone derivative 11 shown in FIG. 6 has many known precedents (17). The present inventors have carried out many such coupling reactions in their laboratory and have found that molecules containing active hydrogens (NH₂ or OH groups) can be successfully coupled in such reactions if care is taken to form the reactive Cu-TMS acetylene complex before the halogenated aromatic substrate is added. It is therefore anticipated that this reaction will proceed as shown in FIG. 6. If, however, the reaction fails to be successful as shown in FIG. 6, the intermediate 4 will be coupled with trimethylsilylacetylene in 9:1 CH₃CN/H₂O catalyzed with Pd(AcO)₂/PPh₃/CuI. The present inventors have carried out a model reaction in their laboratory with an unprotected iodophenol that gave the desired coupling product with this procedure.

FIG. 7 outlines the reaction sequence that will be employed to prepare the 2,3-methylenedioxyestrone derivative (analogue 18). This reaction sequence is based upon the reaction sequence employed by Stubenrauch and Knuppen to prepare catechol estrogens. (18)

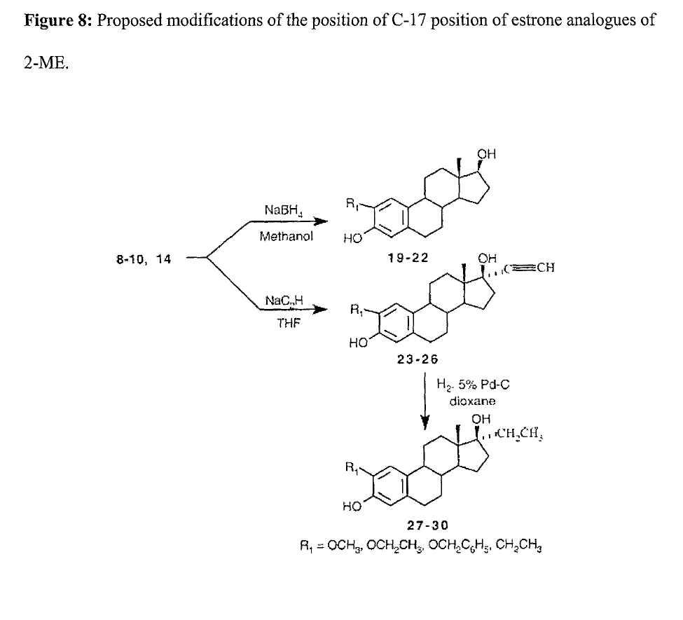

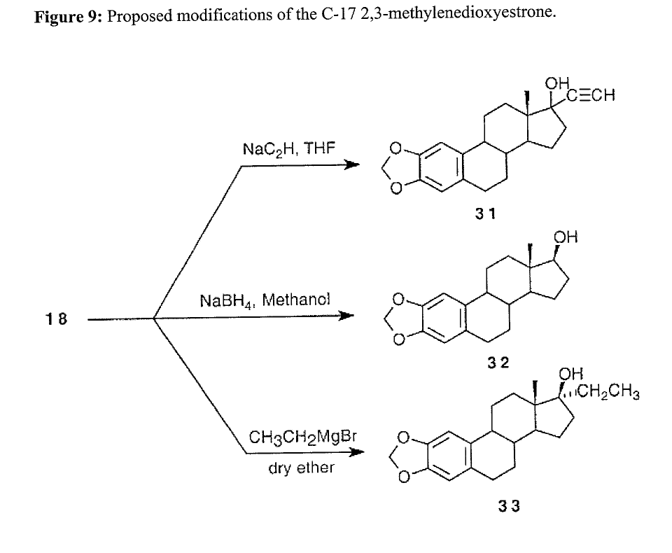

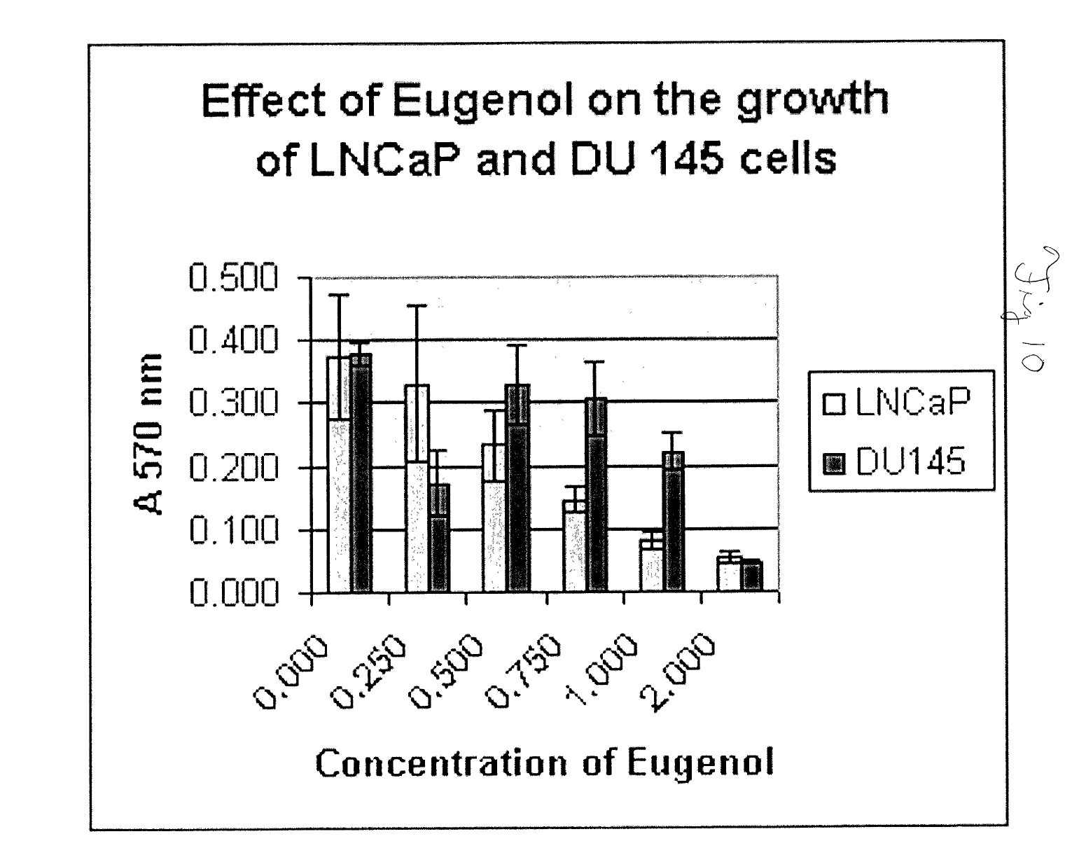

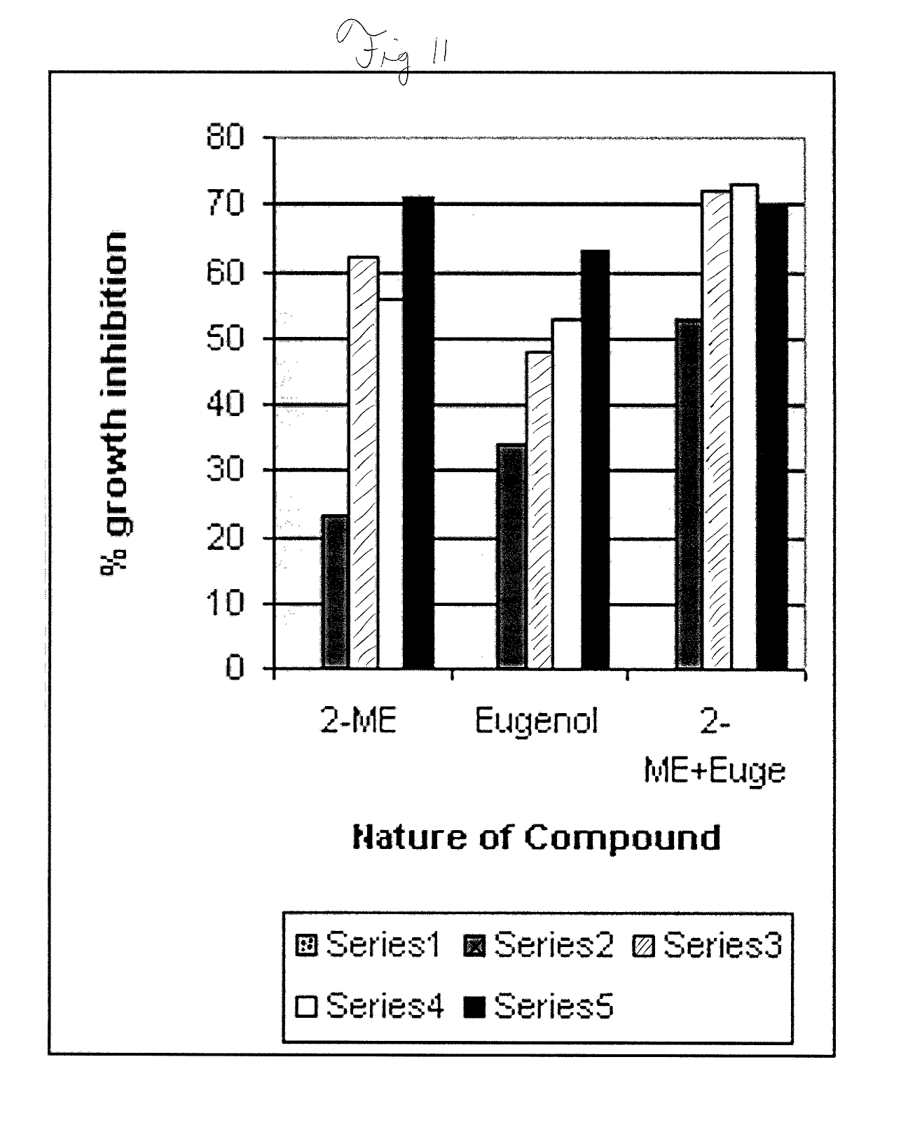

FIGS. 8 and 9 illustrate how 2-methoxyestrone and the 2methoxyestrone analogues prepared as outlined in FIGS. 6 and 7 above will be converted into (i) 2-methoxyestrone and its analogues and (ii) 2, 3-methylenedioxyestrone analogues modified at position C-17. The preparation of these structures will not only allow us to test the requirement for the 17b-hydroxyl group in the chemopreventive activity of 2-ME but will also enable us to determine if substitutions at C-17 (for example, the 17-ethynyl-2-ME derivative, 23) will decrease the rate of metabolism and deactivation of 2-ME and its analogues. As outlined in FIGS. 8 and 9 below, the present inventors propose to prepare both 2-ethyl-17b-estradiol (analogue 22) and 2,3methylenedioxy-17b-estradiol (analogue 32). In addition, since 17a-ethynylestradiol (ethynylestradiol) is both a potent estrogenic and long-lived analogue of E₂, the 17a-ethynyl derivative of 2-ME (analogue 19) will be prepared as outlined in FIG. 8. In addition, by directing synthesis to produce estrone analogues of the target structures (analogues 8-10, 14, and 18) as illustrated in FIGS. 6 and 7, it will be possible to prepare 17a-ethynyl, and 17a-ethyl derivatives of the 2-alkoxy, 2-ethyl, and 2,3-methylenedioxy analogues (analogues 23-26, 27-30, 31 and 32).

It should be noted that the proposed reactions used to modify the C-17 carbonyl of the estrone analogues shown in FIGS. 8 and 9 are standard reactions that have been successfully applied to estrone. (19)

Although not explicitly shown in FIG. 6 and 8, the 2-ethynyl intermediate shown in FIG. 6 (analogue 12) will also be converted into 2-ethynylestrone and 2-ethynylestradiol for testing. Further, although not explicitly indicated in FIGS. 6 and 7, the 2-ethynylestrone derivative 11 shown in FIG. 6 will also be converted into 2-ethynylestrone and 2-ethynylestradiol as shown in FIG. 7 for the other intermediates. This will generate two additional 2-ME analogues for biological testing. Lastly, it is also possible to modify the acetylene coupling reaction shown in FIG. 6 to prepare 2-(1-propynyl) and 2-(1-butynyl) derivatives of 2-ME that could serve as precursors of 2-propyl and 2-butyl 2-ME analogues.

The synthesis reactions in FIGS. 6-9 outlined above will provide an efficient way of generating 2-ME (analogue 19) and fourteen 2-ME analogues (analogues 20-33) that can be utilized to determine the effects of modifying both the C-17 and the C-2 position of 2-ME. Samples of the estrone analogues themselves (analogues 8-10, 14, 18) will also be tested for their potential growth-inhibitory activity. The reaction sequences outlined in FIGS. 6-9 will therefore produce a total of 21 new 2-ME analogues to be tested as potential selective inhibitors of brain and nervous system cancer cell growth and angiogenesis. It is anticipated that one or more of these analogues may manifest selective growth-inhibitory activities towards cancer cells while, at the same time, being less subject to metabolic conversions that will deactivate or eliminate these active analogues. It is also likely that 17a-ethynyl derivative of 2-ME may have a longer effective half-life both in vitro and in vivo.

### References

1. Gelbke, H. P., and Knuppen, R. 1976. The exertion of five different 2-hydroxyestrogen monomethyl ethers in human pregnancy urine. J Steroid Biochem. 7: 457-463.

2. Zhu, B. T. and Conney, A. H. 1998. Is 2-methoxyestradiol an endogenous estrogen metabolite that inhibits mammary carcinogenesis. Cancer Res. 58: 2269-2277.

3. Fotsis, T., Zhang, Y., Pepper, M. S., Adlercreutz, H., Montesano, R., Nawroth, P. P. and Schweigerer, OL. 1994. The endogenous estrogen metabolite 2-methoxyestradiol inhibits angiogenesis and suppresses tumor growth. Nature. 368: 237-239.

4. Kumar, A. P. and Slaga, T. J. 2000. Weel-cdc2 pathway: A novel target for prostate cancer chemoprevention by an estrogen metabolite. Under review for publication in PNAS (revision).

5. Krishan, A. 1975. Rapid flow cytofluorometric analysis of mammalian cell cycle by propodium iodide staining. J. Cell. Biol. 66:188-193.

6. Zamai, L., Falcieri, E., Zauli, G., Cataldi, A. and Vitale, M. 1993. Optimal detection of apoptosis by flow cytometry depends on cell morphology. Cytometry. 14:891-897.

7. Kerr, J. F. R. and Harmon, B. V. 1991. Apoptosis: The molecular basis of cell death. Cold Spring Harbor Press, New York.

8. Wyllie, A. H. 1980. Glucocortoroid-induced thymocyte apoptosis is associated with endogenous endonuclease activation. Nature. 284: 555-556.

9. Bradford, M. M. 1976. A rapid and sensitive method for the quantitation of microgram quantities of protein utilizing the principle of protein dye binding. Anal. Biochem. 72:284-254.

10. Thornberry, N. A. 1999. Caspases: A decade of death research. Cell Death and Differentiation. 6:1023-1027.

11. Laemmli, U. K. 1970. Cleavage of structural proteins during the assembly of head bacteriophage T4. Nature. 227:680-685.

12. Kaelin, W. G. 1999. The emerging p53 family. J. Natl. Cancer Inst. 91:594-598.

13. El-Deiry, W. S. 1998. Regulation of p53 downstream genes. Seminars in Cancer Biol. 8:345-357.

14. Jost, C., Marin,M. and Kaelin, W. G. 1997. p73 is a human p-53 related protein that can induce apoptosis. Nature. 389:191-194.

15. Yang, A., Kaghad, M., Wang, Y., Gillett, E., Fleming, M. D., Dotsch, V. et al. 1998. p63, a p53-related protein, p73L, with high homology to p73. Biochem. Biophys. Res. Commun. 248:603-607.

16. Ikegawa, S., Kurosawa, T., and Tohma, M. (1988) Syntheses of C-2 caecholic equilin and equilin derivatives for use in metabolic studies. Chem. Pharm Bull. 36:2993-2999.

17. Neenan, T. X., and Whitesides, G. M. (1988) Synthesis of high carbon monomers bearing multiple ethynyl groups. J. Org. Chem., 53:2489-2496.

18. Stubenrauch, G. And Knuppen, R. (1976) Convenient large scale preparation of catechol estrogens. Steroids, 28:733-741.

19. Fieser, L. F. And Fieser, M. (1959) Estrogens in Steroids, Chapter 15, 444-502, Chapman and Hall, Ltd. London.

We claim:

1. A method for inhibiting cancerous cell proliferation comprising the steps of:
selecting a composition containing 2-methoxyestradiol, the 2-ethyl-17-β-estradiol molecules identified as analogues 20-22 in FIG. 8, the 17-α-ethynyl molecules identified as analogues 23-26 in FIG. 8, the 17-α-ethyl molecules identified as analogues 27-30 in FIG. 8, the 2,3-methylenedioxy molecules identified as analogues 31, 32, and 33 in FIG. 9, the 2-alkoxy substituted analogues of estrone molecules identified as analogues 8-10 in FIG. 6, the 2-ethyl substituted molecule identified as analogue 14 in FIG. 6, or the 2,3-methylenedioxyestrone molecule identified as analogue 18 in FIG. 7; and
administering said composition to cells in which is identified suspected cancer cells.

2. The method of claim 1 wherein said suspected cancer cells are brain cancer cells.

3. The method of claim 1 wherein said suspected cancer cells are nervous system cancer cells.

4. The method of claim 1 wherein said suspected cancer cells are brain cancer cells and nervous system cancer cells.

5. A method for inhibiting cancerous cell proliferation comprising the steps of:
selecting a composition consisting substantially of one or more of 2-methoxyestradiol, 2-ethoxyestradiol, 2-butoxyestradiol, 17-α″-ethynylestradiol with methoxy group at position 2, 17-″α-ethynylestradiol with butoxy group at position 2, 17-α″-ethynyl-9-α″-fluoroestradiol with methoxy group at position 2; and 17-α″-ethynyl-9-″α-fluoroestradiol with butoxy group at position 2; and
administering said composition to cells in which is identified suspected cancer cells.

5. The method of claim 5 wherein said suspected cancer cells are brain cancer cells.

6. The method of claim 5 wherein said suspected cancer cells are nervous system cancer cells.

7. The method of claim 5 wherein said suspected cancer cells are brain cancer cells and nervous system cancer cells.

8. A composition for application to cancerous cells consisting in active constituents substantially of one or more agents chosen from a group consisting of 2-methoxyestradiol, 2-ethoxyestradiol, 2-butoxyestradiol, 17-α″-ethynylestradiol with methoxy group at position 2, 17-″α-ethynylestradiol with butoxy group at position 2, 17-α″-ethynyl-9-α″-fluoroestradiol with methoxy group at position 2, and 17-α″-ethynyl-9-″α-fluoroestradiol with butoxy group at position 2, 2-ethyl-17-β-estradiol molecules identified as analogues 20-22 in FIG. 8, the 17-α-ethynyl molecules identified as analogues 23-26 in FIG. 8, the 17-α-ethyl molecules identified as analogues 27-30 in FIG. 8, the 2,3-methylenedioxy molecules identified as analogues 31, 32, and 33 in FIG. 9, the 2-alkoxy substituted analogues of estrone molecules identified as analogues 8-10 in FIG. 6, the 2-ethyl substituted molecule identified as analogue 14 in FIG. 6, or the 2,3-methylenedioxyestrone molecule identified as analogue 18 in FIG. 7.

9. The method of claim 8 wherein said suspected cancer cells are brain cancer cells.

10. The method of claim 8 wherein said suspected cancer cells are nervous system cancer cells.

11. The method of claim 8 wherein said suspected cancer cells are brain cancer cells and nervous system cancer cells.
